(19) United States
(12) Patent Application Publication
(10) Pub. No.: US 2014/0161847 A1
(43) Pub. Date: Jun. 12, 2014

(54) BREATH FRESHENING PRODUCT

(71) Applicant: 32 ORAL CARE, LLC, New York, NY (US)

(72) Inventor: Thomas P. Connelly, Hopkinton, MA (US)

(73) Assignee: 32 ORAL CARE, LLC, New York, NY (US)

(21) Appl. No.: 13/875,693

(22) Filed: May 2, 2013

Related U.S. Application Data
(60) Provisional application No. 61/735,849, filed on Dec. 11, 2012.
(60) Provisional application No. 61/811,261, filed on Apr. 12, 2013.

(51) Int. Cl. A61K 8/92; A61K 8/36; A61Q 11/00; A61K 8/34

(52) U.S. Cl. CPC A61K 8/922; A61K 8/345; A61K 8/36; A61Q 11/00
USPC 424/401; 424/58

(57) ABSTRACT

An improved method and system for reducing the condition of bad breath is provided. The system is directed to a breath freshening composition which includes a plurality of sugar alcohol carbonated crystals as a delivery medium, to which a vegetable or nut oil, and a zinc based compound for controlling bad breath are added. The composition is prepared by first producing the sugar alcohol carbonated crystals and then adding to said crystals, preferably by spraying, the vegetable or nut oil and the zinc based compound. Additional ingredients may be added to the carbonated crystals, including sugar based alcohols for inhibiting bacterial growth, flavor powders and sweeteners.

### RELATED APPLICATION(S)

This application is based on and claims priority to U.S. Provisional Patent Application No. 61/735,849 filed on Dec. 11, 2012, and U.S. Provisional Patent Application No. 61/811,261 filed on Apr. 12, 2013, both of which are hereby incorporated herein by reference.

### FIELD OF THE INVENTION

This invention is directed to a breath freshening product or composition.

### BACKGROUND OF THE INVENTION

Bad breath has been a documented concern since ancient Greek and Roman times, and continues to be a problem today. Bad breath comes in two common forms: intrinsic and extrinsic. Intrinsic oral malodor (bad odor) is most commonly caused by substances produced by bacteria in the oral cavity (your mouth). Anaerobic bacteria found in the rear folds of the tongue are believed to generate VSCs (volatile sulfur compounds), which cause bad breath. This happens during the day as one eats, but is also especially common at night, when one produces less saliva (causing your classic “morning breath”). This is why the best time to clean one's mouth is before bed.

Extrinsic oral malodor originates from the ingestion of substances (such as cheese, garlic, tobacco) that contain malodorous compounds. The duration of extrinsic malodor is proportional to the amount and frequency of ingestion of the offending substances and the amount of time required to ‘wash-out’ the malodorous substances.

The human nose is incredibly sensitive to sulfur. This explains why very low levels of sulfur compounds are added to otherwise colorless and almost odorless natural gas so that leaks can be detected before a fire or explosion occurs. Unfortunately, one's mouth makes sulfur compounds all day. This is why “bad breath” is easy for others to detect.

Accordingly, it would be desirable to provide an improved system and method for ameliorating the condition of bad breath.

### SUMMARY OF THE INVENTION

Generally speaking, in accordance with the invention, an improved composition for alleviating and treating the condition of bad breath is provided. The system is directed to a breath freshening product which includes a plurality of sugar alcohol carbonated crystals as a delivery medium, to which a vegetable or nut oil and a zinc based compound for controlling bad breath are added. The product is prepared by first producing the sugar alcohol carbonated crystals and then adding to said crystals, preferably by spraying, the vegetable or nut oil and the zinc based compound.

Additional ingredients may be added to the carbonated crystals, including sugar alcohols for inhibiting bacterial growth, flavor powders and sweeteners.

Accordingly, it is an object of the invention to provide an improved breath freshening product.

Another object of the invention is to provide a breath freshening product that is easy to use.

A further object of the invention is to provide a breath freshening product which also inhibits bacterial growth.

Yet another object of the invention is to provide a breath freshening product which rejuvenates tooth enamel.

Still other objects and advantages will be apparent from the following description.

### DETAILED DESCRIPTION OF THE INVENTION

The inventive breath freshening product includes a plurality of sugar alcohol carbonated crystals/rocks as the delivery medium. The sugar alcohol crystals may be present in an amount between about 10 and 99 weight percent of the overall composition. Preferably, the crystals are present in an amount between about 90 and 95 weight percent. Each crystal may be between about 1 and 40 mm in diameter in size, with between about 4 and 20 mm being preferred. The preferred sugar alcohol crystals are isomalt carbonated crystals; others that may be used include mannitol carbonated crystals, xylitol carbonated crystals, sorbitol carbonated crystals and glycerol carbonated crystals.

The inventive product also includes vegetable or nut oil. The oil may be present in an in an amount no greater than about 10 weight percent and should be present in an amount in the overall composition between about 0.1 and 10 weight percent, with between about 0.48 and 10 weight percent being preferred. The preferred oil is sunflower oil, although other oils such as olive oil and linseed oil can be used. The purpose of the oil in the inventive product is to prevent caking. Moreover, if flavor and artificial sweeteners are added to the inventive formulation, the oil component facilitates the blending of these two ingredients. The vegetable or nut oil is added to the carbonated crystals, preferably by being sprayed thereon.

The inventive product also includes a zinc based bad breath controlling ingredient, which is preferably in powder form. The zinc based compound is a zinc salt, preferably water soluble, and may be present in the overall composition in an a amount no greater than about 10 weight percent and should be present in an amount between about 0.1 and 10 weight percent, with between about 0.5 and 10 weight percent being preferred. The preferred bad breath controlling ingredient is zinc gluconate, although zinc chloride, zinc sulfate and zinc citrate, as well as other salts which chemically interact with volatile sulphur compounds (VSCs—bad breath gases) may be used. (The zinc ions bind the precursor compounds that bacteria require in order to make VSCs). The bad breath controlling ingredient promotes pleasant breath as well as preventing tarter formation. This ingredient is also added to the carbonated crystals preferably by spraying.

The inventive product may also include a non-crystalline sugar alcohol, preferably in powder form. The sugar alcohol may be present in the overall composition in an amount no greater than about 10 weight percent and should be present in an amount between about 1 and 10 weight percent. Sugar alcohols inhibit the ability of bacteria in the mouth to attach to tooth enamel and thereby cause tooth decay. The preferred sugar alcohol is xylitol. Other sugar alcohols include sorbitol and mannitol. This ingredient is added to the carbonated crystals, preferably by spraying.

Optionally, the inventive product includes a flavor, preferably as a powder. The flavor may be present in the overall composition in an amount no greater than 10 weight percent and should be in an amount between about 1 and 10 weight percent. The preferred flavor powder is peppermint. Others include cinnamon, wintergreen, spearmint, orange mint, fruit, mint, cherry, orange and strawberry. The flavor should be suitable for spray application to the carbonated crystals.

Optionally, the inventive product includes an artificial sweetener, preferably in powder form. The artificial sweetener may be present in the overall composition in an amount no greater than 10 weight percent and should be present in an amount between about 0.01 and 10 weight percent, with between about 0.02 and 10 weight percent being preferred. The preferred artificial sweetener is sucralose. Other suitable artificial sweeteners include saccharin, aspartame and stevia. The sweetener should be suitable for spray application.

Optionally, the inventive product includes an edible enamel remineralizer that is available under the trademark Rennou from Theocorp Holding Company, LLC of Metairie, La., and is present in the overall composition in an amount between about 0.1 and 75 weight percent. Rennou is a non-flouride, cocoa derived, human tooth enamel remineralizer (currently found in Theodent brand toothpaste).
A preferred formulation of the inventive product is as follows:

92.00% Isomalt Carbonated Crystals, size 04-20 mm

02.00% Spray Dried Peppermint Flavor Powder

00.48% Sunflower Oil (processing aid)

00.02% Sucralose Powder

00.50% Zinc Gluconate Powder

05.00% Xylitol Powder
Other formulations are as follows:

91.50% lsomalt Carbonated Crystals size 04-20 mm

02.00% Spray Dried Peppermint Flavor Powder

00.48% Sunflower Oil (processing aid)

00.02% Sucralose Powder

01.00% Zinc Gluconate Powder

05.00% Xylitol Powder

90.50% lsomalt Carbonated Crystals, size 04-20 mm

02.00% Spray Dried Peppermint Flavor Powder

00.48% Sunflower OH (processing aid)

00.02% Sucralose Powder

02.00% Zinc Gluconate Powder

05.00% Xylitol Powder

91.50% lsomalt Carbonated Crystals, size 04-20 mm

02.00% Spray Dried Peppermint Flavor Powder

00.48% Sunflower Oil (processing aid)

00.02% Stevia Powder

01.00% Zinc Gluconate Powder

05.00% Xylitol Powder

90.50% Isomalt Carbonated Crystals, size 04-20 mm

02.00% Spray Dried Peppermint Flavor Powder

00.48% Sunflower OH (processing aid)

00.02% Stevia Powder

02.00% Zinc Gluconate Powder

05.00% Xylitol Powder

In order to prepare the inventive product, isomalt or another sugar alcohol is first heated to a liquid state, the liquid isomalt or other sugar alcohol is then gasified with pressurized carbon dioxide, and the isomalt or other sugar alcohol is allowed to cool and harden. Once the isomalt or other sugar alcohol is cooled to a solid state, it is fractured into random sizes of solid isomalt “rocks”. These rocks are then screened for size. The final rocks/crystals are sprayed with a mixture of the oil, bad breath controlling ingredient (zinc based compound), xylitol (or other sugar alcohol powder), flavor and sweetener.

Alternatively, instead of spraying, one could bake the powder ingredients into the crystals.

In order to use the inventive product, crystals made in accordance with the invention are poured into one's mouth and then are swished in the mouth for approx. 60 seconds. The crystals should then be rubbed against one's teeth using one's tongue or cheeks. Thereafter, the crystals can be swallowed. This type of application will help sustain fresh breath in one's mouth.

The formulation of the invention has been shown to inhibit odiferous volatile sulphur compounds, effectively eliminating the cause of bad breath.

In testing, twenty patients between the ages of 22 and 67 were studied. The study spanned two days. On day one, the patient's breath was measured for the presence of volatile sulfur compounds (VSCs) at four intervals: 2 pm, 3 pm, 4 pm and 5 pm. The patients were not allowed to drink or eat from 10 am unto the final measurement at 5 pm. On day two, the patients were treated at 1 pm with a packet of crystals made in accordance with the preferred inventive formula. Each patient's breath was remeasured for the presence of VSCs at the same intervals: 2 pm, 3 pm, 4 pm and 5 pm. The patients were not allowed to drink or eat from 10 am until the final measurement at 5 pm. The method used to measure the presence of VSCs was an Interscan Halimeter calibrated to 0 parts per billion (ppb) prior to the study,
Bad breath is associated with VSC levels as follows:
50-190 ppb=normal
150-300 ppb=noticeable breath
300-500 ppb=bad breath
>500 ppb=rare bad breath (extreme)
On day one, the measured amount of VSCs (in ppb) among the 20 patients that were rested is listed in the table below:

DAY 1 (all numbers in ppb)

| Patient | VSC 2 pm | VSC 3 pm | VSC 4 pm | VSC 5 pm |
|---|---|---|---|---|
| 1 | 307 | 311 | 344 | 349 |
| 2 | 140 | 155 | 155 | 202 |
| 3 | 411 | 409 | 443 | 420 |
| 4 | 212 | 222 | 234 | 278 |
| 5 | 23 | 55 | 56 | 44 |
| 6 | 409 | 410 | 444 | 450 |
| 7 | 291 | 330 | 333 | 343 |
| 8 | 377 | 380 | 388 | 390 |
| 9 | 332 | 378 | 354 | 322 |
| 10 | 290 | 290 | 290 | 294 |
| 11 | 82 | 100 | 106 | 101 |
| 12 | 25 | 44 | 50 | 51 |
| 13 | 308 | 380 | 392 | 393 |
| 14 | 313 | 323 | 319 | 303 |
| 15 | 450 | 456 | 455 | 470 |
| 16 | 231 | 243 | 243 | 245 |
| 17 | 190 | 198 | 201 | 195 |
| 18 | 101 | 91 | 123 | 127 |
| 19 | 444 | 456 | 431 | 402 |
| 20 | 321 | 345 | 362 | 354 |

On day two, each of the tested patients used a packet of crystals at 1 pm made in accordance with the invention. The patients were then remeasured for the presence of VSCs in ppb. The results are in the table below.

DAY 2

| Patient | VSC 2 pm | VSC 3 pm | VSC 4 pm | VSC 5 pm |
|---|---|---|---|---|
| 1 | 101 | 122 | 141 | 178 |
| 2 | 72 | 113 | 112 | 149 |
| 3 | 133 | 137 | 187 | 191 |
| 4 | 87 | 107 | 107 | 113 |
| 5 | 9 | 18 | 22 | 12 |
| 6 | 122 | 131 | 141 | 142 |
| 7 | 154 | 151 | 157 | 167 |
| 8 | 102 | 109 | 107 | 111 |
| 9 | 74 | 132 | 140 | 135 |
| 10 | 137 | 137 | 145 | 151 |
| 11 | 19 | 21 | 77 | 56 |
| 12 | 7 | 25 | 26 | 31 |
| 13 | 111 | 137 | 143 | 155 |
| 14 | 167 | 171 | 162 | 141 |
| 15 | 143 | 176 | 153 | 149 |
| 16 | 111 | 131 | 144 | 179 |
| 17 | 101 | 120 | 131 | 159 |
| 18 | 34 | 44 | 62 | 98 |
| 19 | 176 | 191 | 202 | 202 |
| 20 | 190 | 188 | 191 | 193 |

In comparing the VSC measurements in the twenty patients from day one to day two, a statistically significant reduction of the presence of VSCs in all twenty patients was found. The average VSC measurement on day one was 278 ppb, well within the spectrum of noticeable bad breath. After treatment on day two with a packet of crystals made in accordance with the invention, the average VSC measurement on day two was 120 ppb, well within the normal range of VSCs and considered to be pleasant breath.

The test data demonstrate that breath treatment in accordance with the invention not only removes malodor, the unique delivery system and active ingredients therewithin significantly reduces the reappearance of malodor over the course of many hours.

The scope of the invention will now be defined in the following claims.

1. A breath freshening composition comprising:
a plurality of sugar alcohol carbonated crystals;
a vegetable or nut oil; and
a zinc salt.

2. The composition of claim 1, wherein the crystals are present in the overall composition in an amount between about 10 and 99 weight percent.

3. The composition of claim 1, wherein the crystals are selected from the group consisting of isomalt carbonated crystals, mannitol carbonated crystals, xylitol carbonated crystals, sorbitol carbonated crystals and glycerol carbonated crystals.

4. The composition of claim 1, wherein each crystal has a diameter of between about 1 and 40 mm.

5. The composition of claim 1, wherein the vegetable or nut oil is present in the overall composition in an amount between about 0.1 and 10 weight percent.

6. The composition of claim 1, wherein the vegetable or nut oil is selected from the group consisting of sunflower oil, olive oil and linseed oil.

7. The composition of claim 1, wherein the zinc salt is present in the overall composition in an amount between about 0.1 and 10 weight percent.

8. The composition of claim 1, wherein the zinc salt is selected from the group consisting of zinc gluconate, zinc chloride, zinc sulfate and zinc citrate.

9. The composition of claim 1, further including a non-crystalline sugar alcohol.

10. The composition of claim 9, wherein the sugar alcohol is selected from the group consisting of xylitol, sorbitol and mannitol.

11. The composition of claim 9, further including one or more ingredients selected from the group consisting of a flavor powder and an artificial sweetener.

12. The composition of claim 11, wherein the flavor powder is peppermint and the artificial sweetener is sucralose.

13. The composition of claim 9, further including an edible enamel remineralizer.

14. The composition of claim 9, wherein said vegetable or nut oil, zinc salt and sugar based alcohol ingredients are sprayed onto said crystals.

15. A breath freshening composition comprising:
a plurality of sugar alcohol carbonated crystals selected from the group consisting of isomalt carbonated crystals, mannitol carbonated crystals, xylitol carbonated crystals, sorbitol carbonated crystals and glycerol carbonated crystals;
a vegetable or nut oil selected from the group consisting of sunflower oil, olive oil and linseed oil; and
a zinc salt selected from the group consisting of zinc gluconate, zinc chloride, zinc sulfate and zinc citrate.

16. The composition of claim 15, wherein the crystals are isomalt carbonated crystals, the oil is sunflower oil and the zinc salt is zinc gluconate.

17. The composition of claim 15, wherein the crystals are present in the overall composition in an amount between about 90 and 95 weight percent, the vegetable oil is present in the overall composition in an amount between about 0.48 and 10 weight percent, and the zinc salt is present in the overall composition in an amount between about 0.5 and 10 weight percent.

18. The composition of claim 15, further including a sugar based alcohol selected from the group consisting of xylitol, sorbitol and mannitol.

19. The composition of claim 15, further including at least one of a flavor powder selected from the group consisting of peppermint, cinnamon, wintergreen, spearmint, orange mint, fruit mint, cherry, orange and strawberry, and an artificial sweetener selected from the group consisting of sucralose, saccharin, aspartame and stevia.

20. The composition of claim 15, wherein the crystals are present in the overall composition in an amount of about 92 weight percent, the vegetable oil is present in the overall composition in an amount of about 0.48 weight percent, and the zinc salt is present in the overall composition in an amount of about 0.50 weight percent.

21. A breath freshening composition comprising:
a plurality of sugar alcohol carbonated crystals present in the overall composition in an amount between about 10 and 99 weight percent;
a vegetable or nut oil present in the overall composition in an amount no greater than about 10 weight percent; and
a zinc salt present in the overall composition in an amount no greater than 10 weight percent.

22. The composition of claim 21, wherein the crystals are selected from the group consisting of isomalt carbonated crystals, mannitol carbonated crystals, xylitol carbonated crystals, sorbitol carbonated crystals and glycerol carbonated crystals, the oil is selected from the group consisting of sunflower oil, olive oil and linseed oil, and the zinc salt is selected from the group consisting of zinc gluconate, zinc chloride, zinc sulfate and zinc citrate.

23. The composition of claim 21, further including a sugar based alcohol in an amount no greater than about 10 weight percent.

24. The composition of claim 21, further including at least one of a flavor powder in an amount no greater than 10 weight percent and an artificial sweetener in an amount no greater than about 10 weight percent

25. A method for treating bad breath comprising the step of administering to a patient an effective amount of a composition comprising a plurality of sugar alcohol crystals, a vegetable or nut oil, and a zinc salt.

26. The method of claim 25, wherein the crystals are selected from the group consisting of isomalt carbonated crystals, mannitol carbonated crystals, xylitol carbonated crystals, sorbitol carbonated crystals and glycerol carbonated crystals, the oil is selected from the group consisting of sunflower oil, olive oil and linseed oil, and the zinc salt is selected from the group consisting of zinc gluconate, zinc chloride, zinc sulphate and zinc citrate.

27. The method of claim 25, wherein said carbonated crystals have a diameter of between about 1 and 40 mm.

28. The method of claim 24, wherein said composition further includes one or more ingredients selected from the group consisting of a sugar alcohol, a flavor powder and an artificial sweetener.

29. The method of claim 28, wherein the crystals are isomalt carbonated crystals; the oil is sunflower oil, and the zinc salt is zinc gluconate.
